(19) United States
(12) Plant Patent Application Publication
(10) Pub. No.: US 2008/0196130 P1
(43) Pub. Date: Aug. 14, 2008

(54) Black berry plant 'Nettleton Creamy White'

(76) Inventors: Scott E. Nettleton, Olney, IL (US); John R. Nettleton, Corona, CA (US)

Correspondence Address: John R. Nettleton, 1125 Country Club Lane, Corona, CA, 92880, US

(21) Appl. No.: 11/510,731

(22) Filed: Aug. 28, 2006

(51) Int. Cl. A01H 5/00

(52) U.S. Cl. PLT/203

(57) ABSTRACT

A new and distinct variety of blackberry plant that in all characteristics looks like a wild blackberry but is distinct in that the fruit when fully ripened is the color is almost a translucent white and not the reddish purple color of the wild blackberry. The new variety named “Nettleton Creamy White” is an upright plant, is a biennial, thorny, with 3 or 5 leaves per plant. The white fruit is medium in size and has the sweetness of its wild blackberry relative.

### BACKGROUND OF THE INVENTION

This new variety of Blackberry plant named the ‘Nettleton Creamy White’ is the result of a spontaneous mutation of the common wild blackberry found in Southern Illinois, which was found on cultivated land near an old homestead in Southern Illinois (Edwards County). The original plant was discovered around 1970. Since that time a few more plants were discovered in 1998 and were transplanted to a garden plot of Scott Nettleton where they have been growing for the past 8 years. The original plants were dug up and transplanted. The new plants are sprouts of new growth coming from the roots of the mother plants. Some of these have been dug up and separated from the mother plant and then replanted in a different location within the same garden plot.

Each plant has the same characteristics as the mother plant, producing the same type of plant with the same type of ‘Creamy White’ fruit.

The ‘Nettleton Creamy White’ is very similar in looks and characteristics to that of the common wild blackberry that grows in Southern Illinois. The plant has upright canes that grow from 5 to 8 feet in height. The green leafy plants have either or both 3 to 5 leaf clusters per plant.

The ‘Nettleton Creamy White’ has biennial canes with a dormant period of the first year canes (primocanes) and the second year canes (floricanes) prior to flowering and fruiting. Once the canes have flowered and produced fruit they die out. The next year's canes are this year's new growth. The canes have 5 distinct sides to them. There are thorns up and down the canes. The plants go dormant in the winter-time and look as thought they were dead. In the Spring-time life returns to the canes and the canes that were last years new growth put on leaves and flowers to produce the berries.

The blooms are similar in size and shape as the wild blackberry having 5 pointed sepals. The only time that it can be determined that the fruit is to be white instead of the normal reddish purple color is after the flowers have bloomed, dropped off and the light green fruit starts fading to a light dull green and then to a green white then at full maturity the fruit is a “Creamy White” an almost translucent color.

The plants bloom at the same time as do the wild Blackberries, which in Southern Illinois is in the end of May and beginning of June. But the fruit of the “Nettleton Creamy White” starts ripening one week sooner then the wild blackberry does, which is in the end of the latter half of June. Each cluster of fruit has an average of 7 berries per cluster.

The “Nettleton Creamy White” blackberry is also growing in a low desert region of Southern California and has been surviving in temperatures of over 105 degrees.

The size of the “Nettleton Creamy White” berries when they are fully ripened averages about ⅝″ to ¾″ in diameter which is a little larger then the wild blackberries. And they have a very sweet taste similar to that of the wild blackberry. Also, the drupelets are like the blackberry and are attached to the center core. When the berries are fully ripe they can be removed from the plant by shaking the canes and the berries will fall off.

### PURPOSED INTENTION OF INVENTION

The purposed “Nettleton Creamy White” blackberry is being grown for the purpose of being propagated and sold through various types of outlets to the general public, plant nurseries and possibly food processing companies as well.

### SUMMARY OF INVENTION

In summary the “Nettleton Creamy White” blackberry is a very unique blackberry in that it has the similar characteristics of the wild blackberries of Southern Illinois but is very distinct in that the berries are a translucent white when they are ripe. The berries when ripe have a sweet distinctive taste though slightly similar to the wild blackberry.

### BRIEF DESCRIPTION OF ILLUSTRATIONS

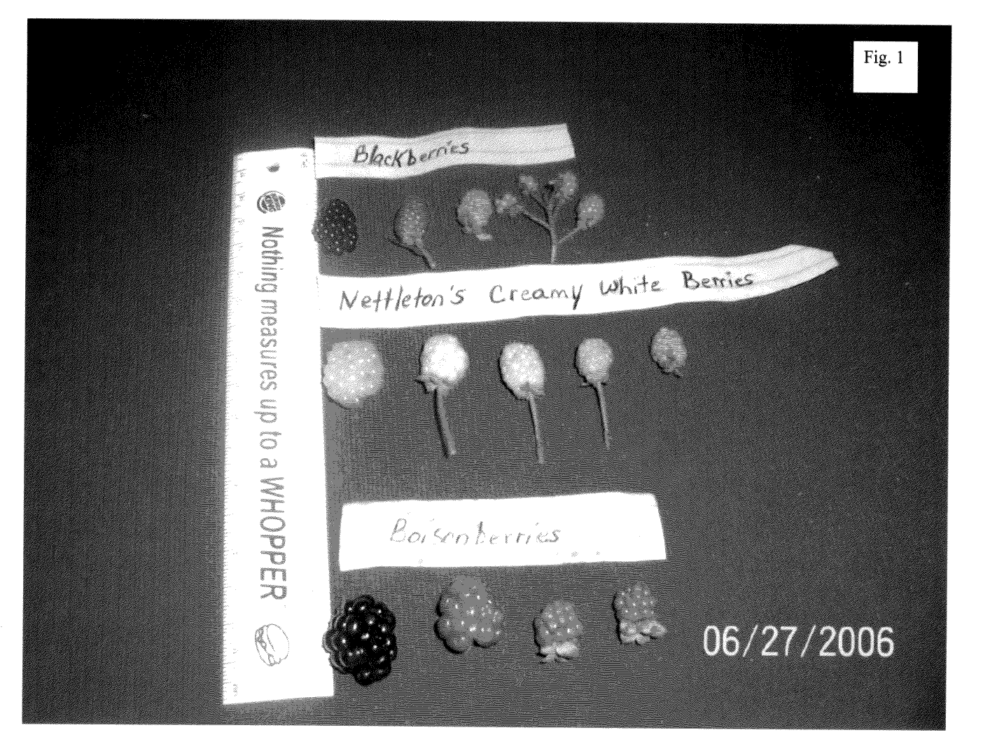

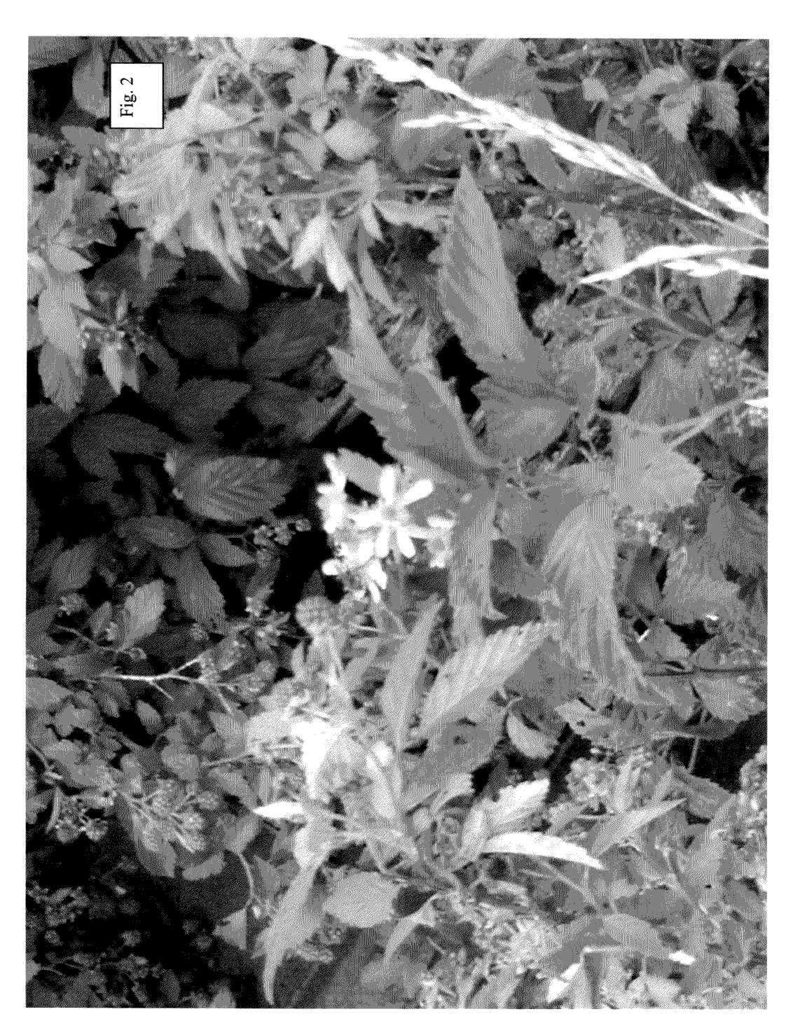

FIG. 1 Comparison of the fruit of wild blackberries, Nettleton Creamy White berries and Boysenberries in the various stages of ripening

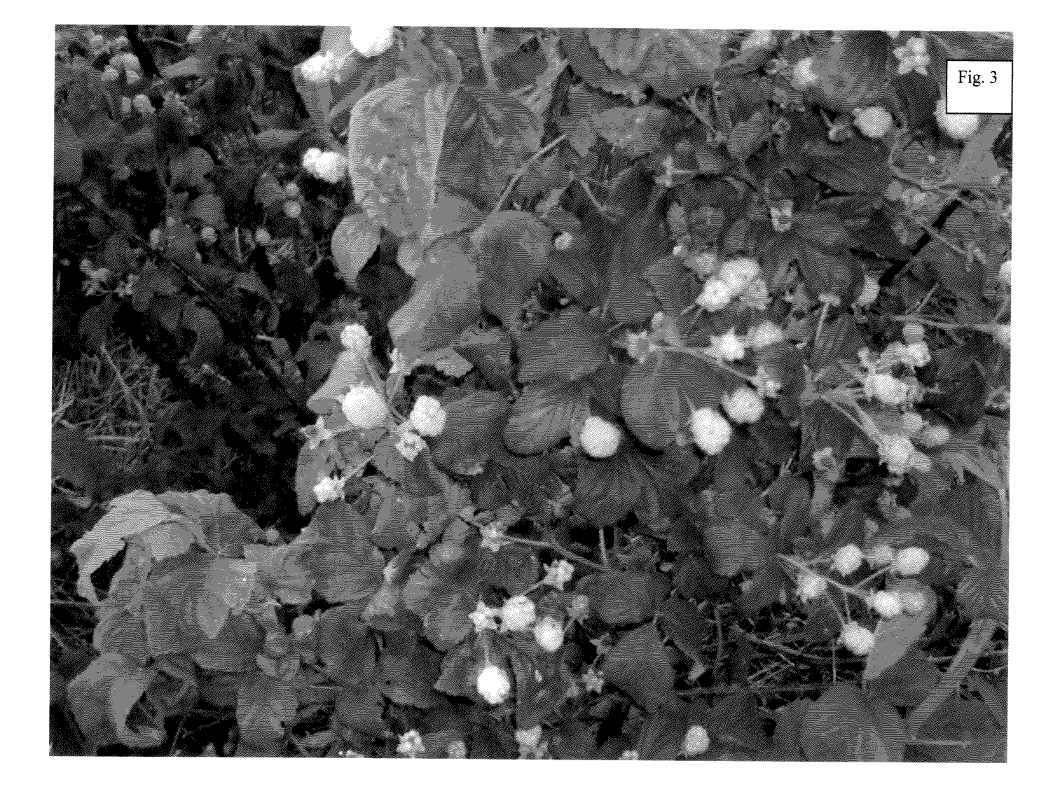

FIG. 2 Southern Illinois wild blackberries growing on the property of Scott Nettleton

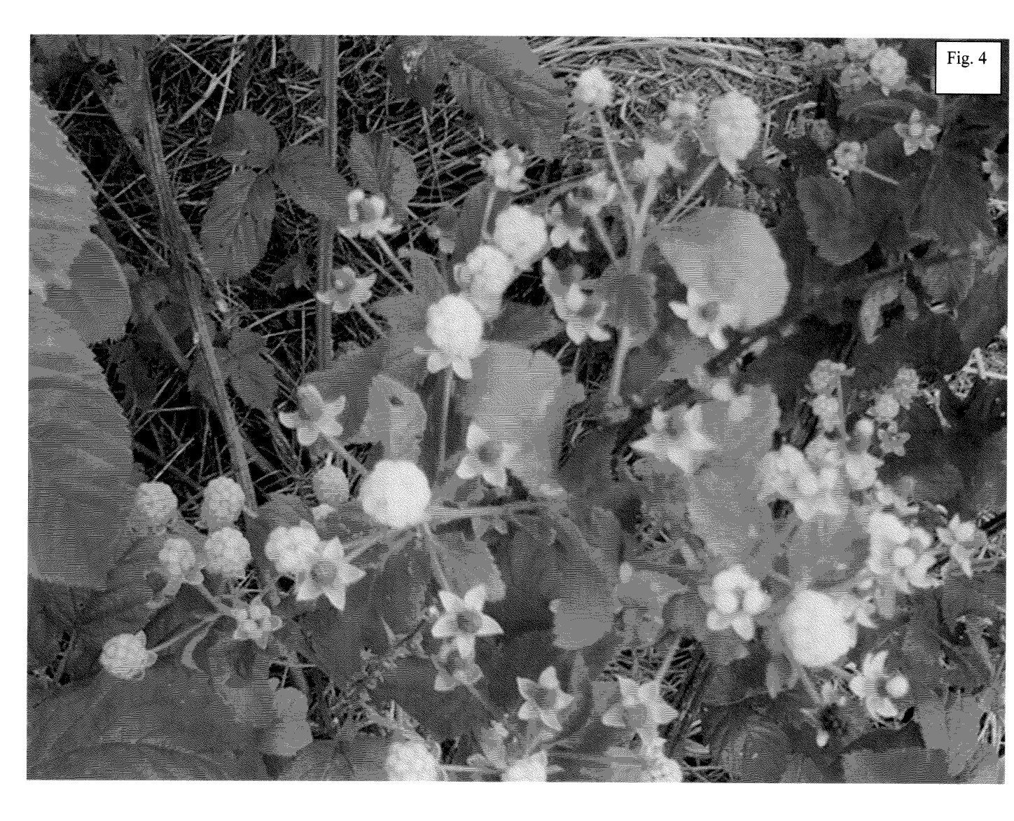

FIG. 3 “Nettleton Creamy White” blackberries in various stages of ripening. Also the size and shape of the sepals can be seen

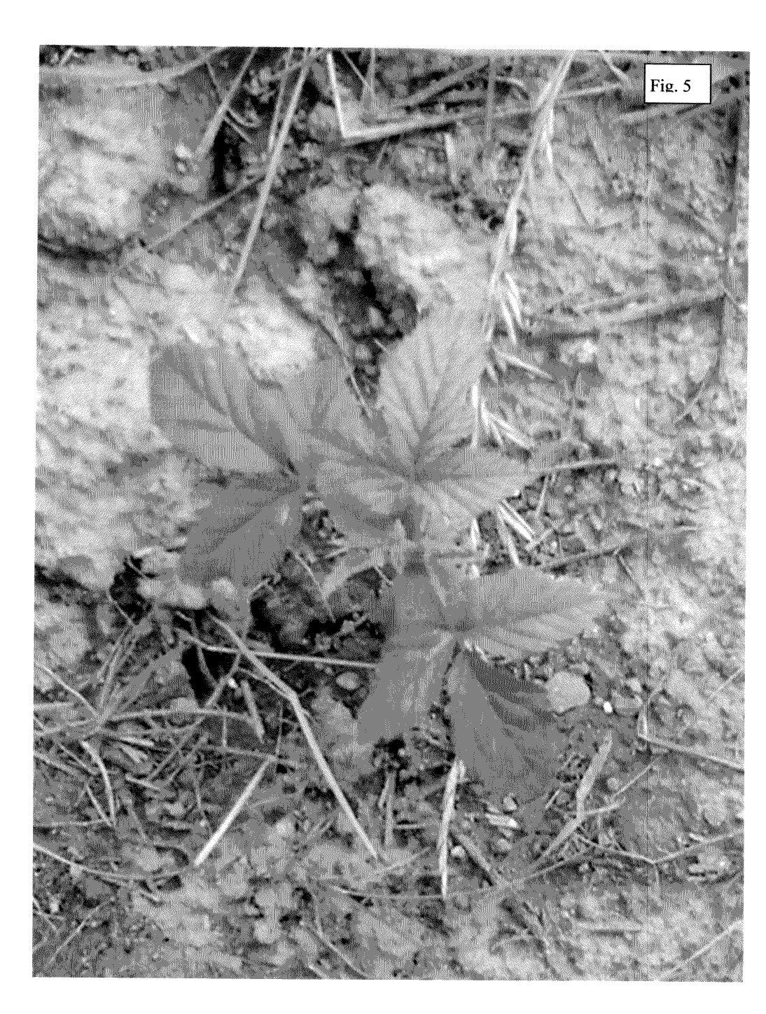

FIG. 4 Close up of “Nettleton Creamy White” blackberry plants showing the Sepals, the Cores, the berries in early and middle stages of ripening

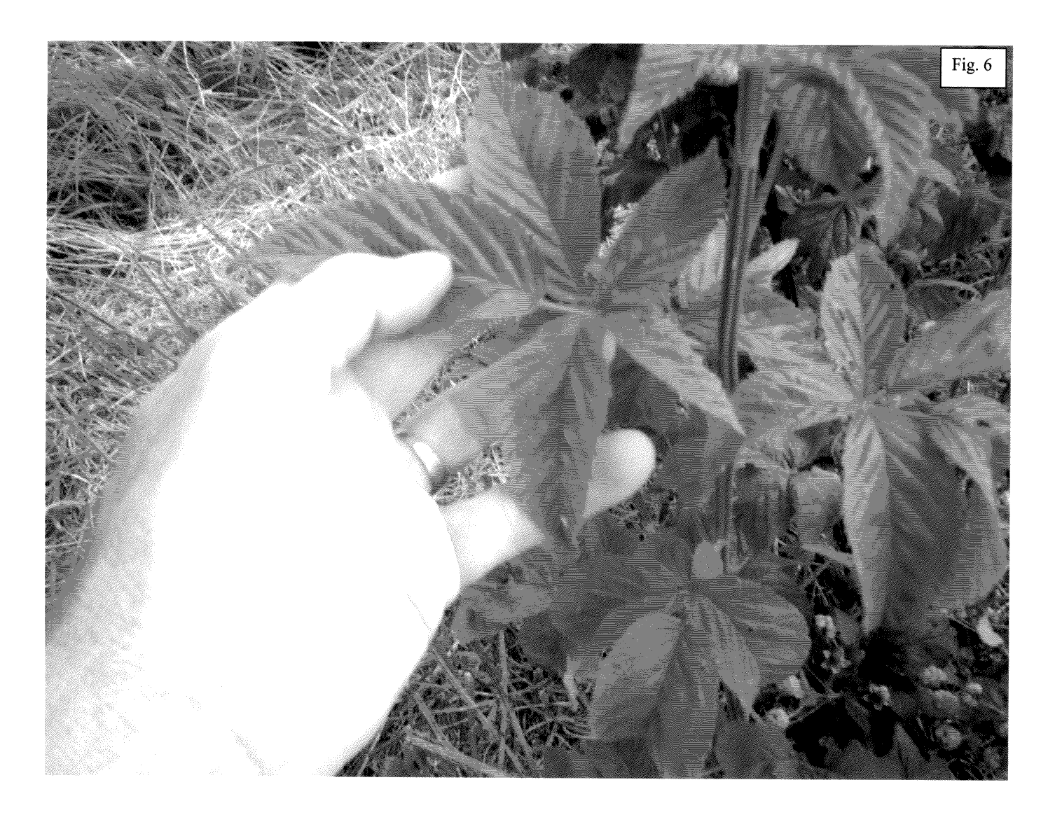

FIG. 5 New plant “Nettleton Creamy White” blackberry

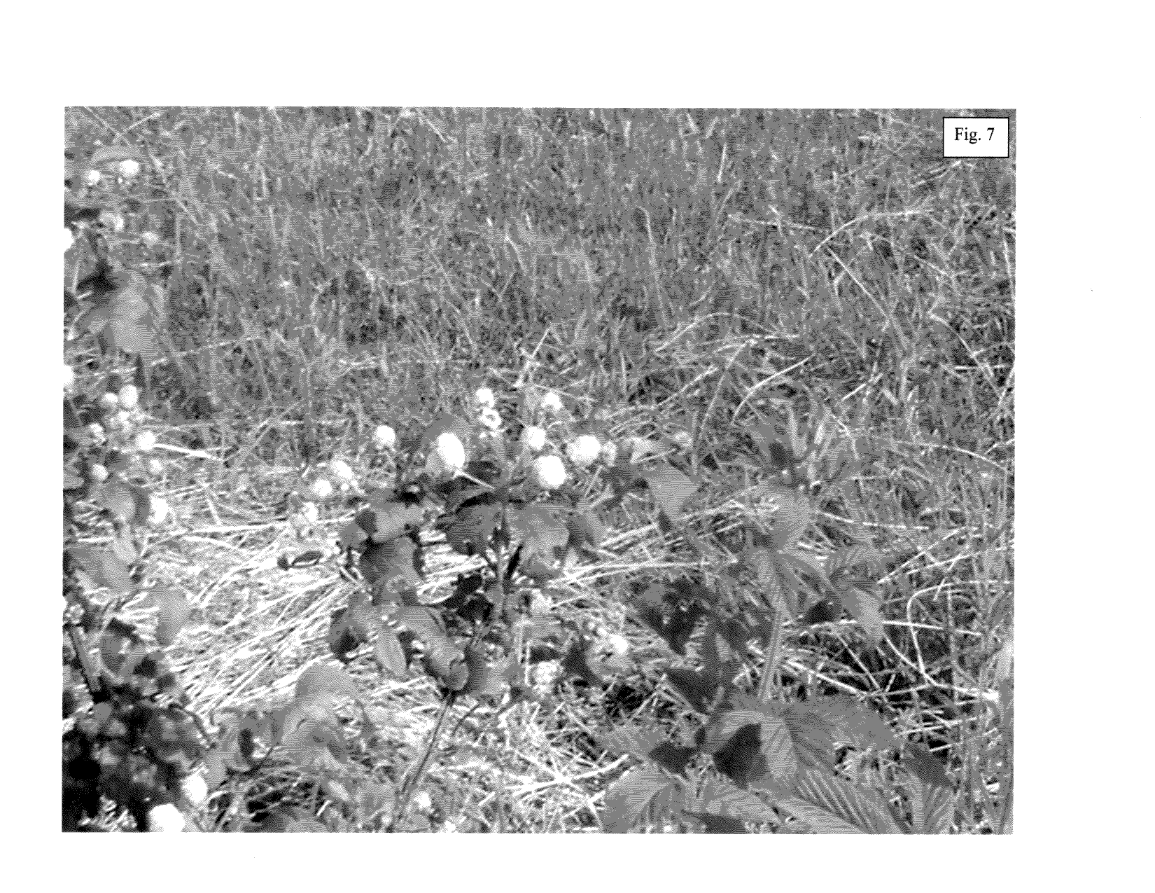

FIG. 6 Close-up of mature plant “Nettleton Creamy White” showing leaves and cane

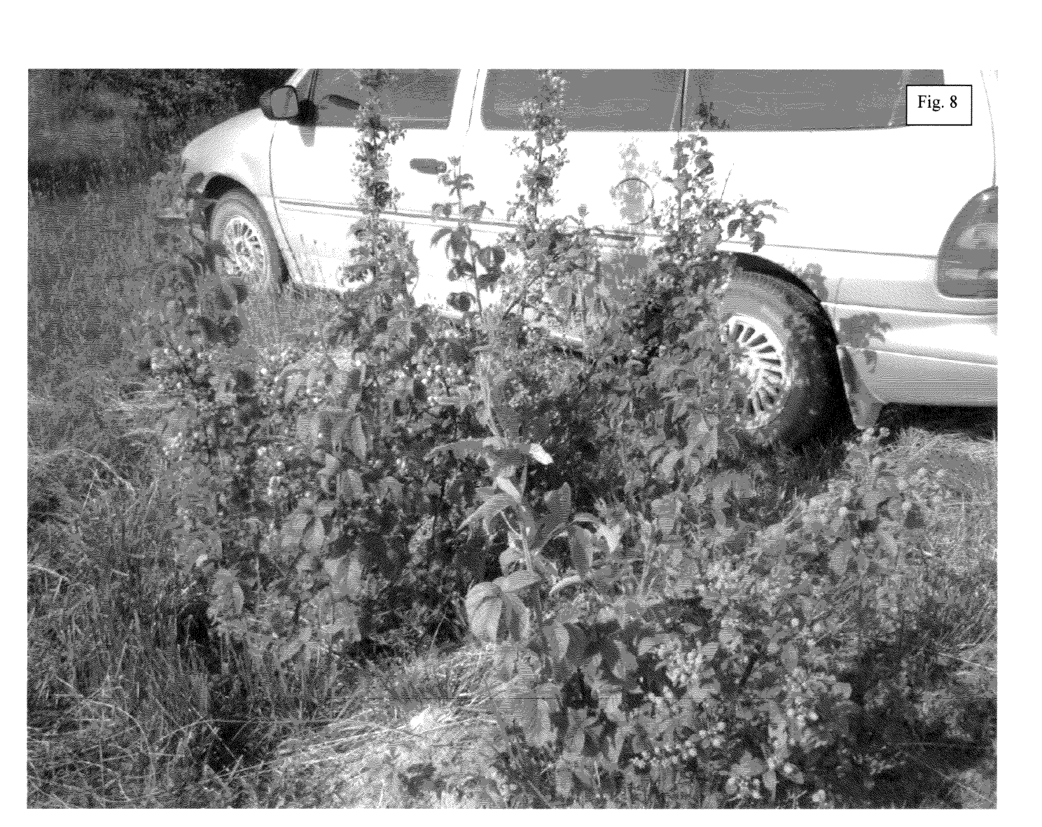

Fig.7 “Nettleton Creamy White” plants showing 2^{nd} year canes with fruit and 1^{st} canes from new growth

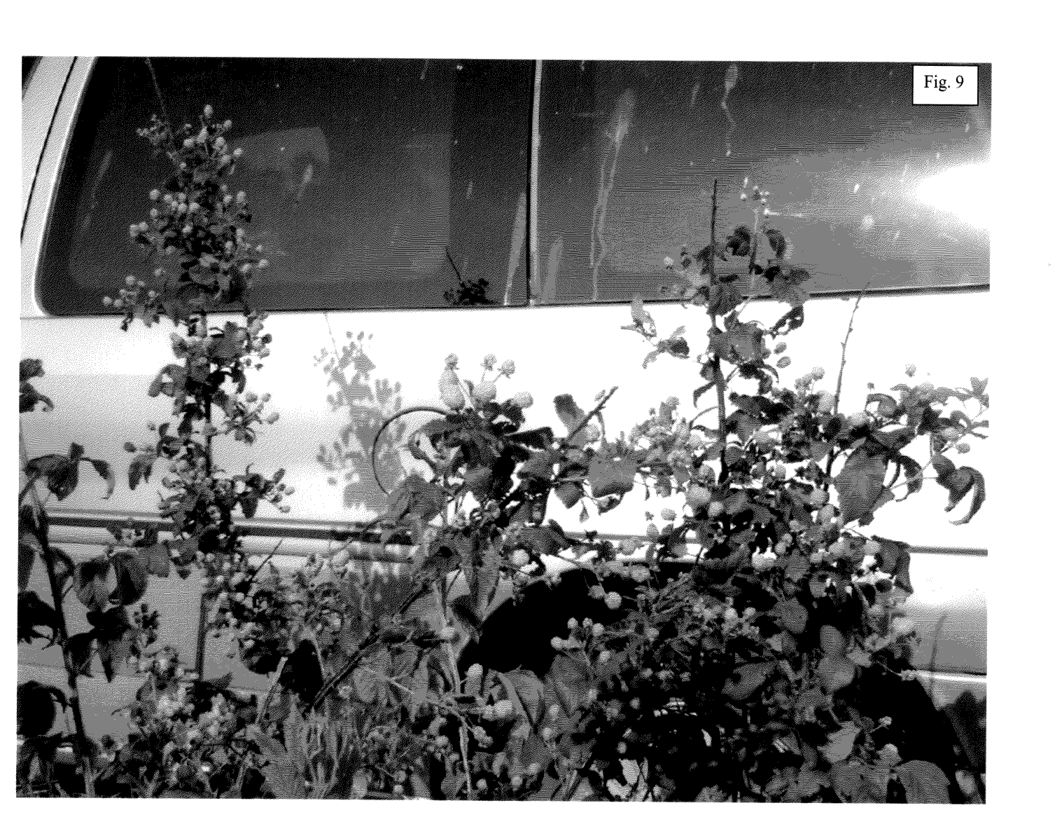

FIG. 8 Small patch of “Nettleton Creamy White” blackberries, 2^{nd} year canes with fruit on them and new 1^{st} year canes

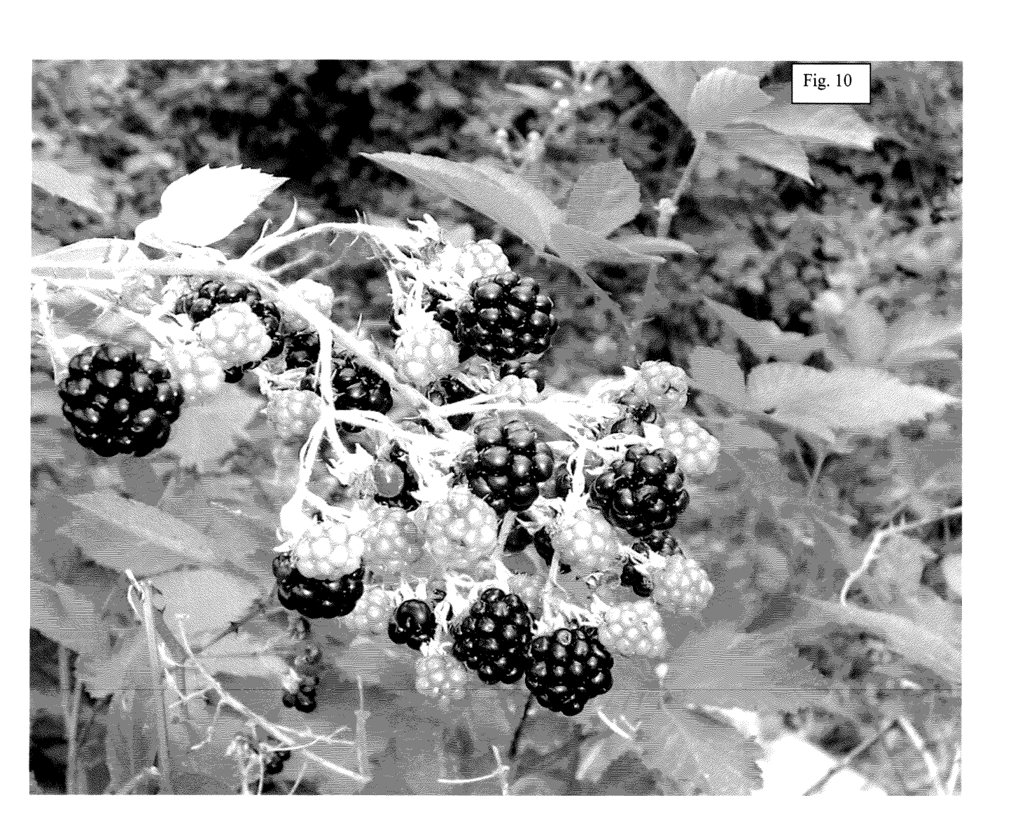

FIG. 9 Showing the quantity of berries produced per each cane. “Nettleton Creamy White” berries

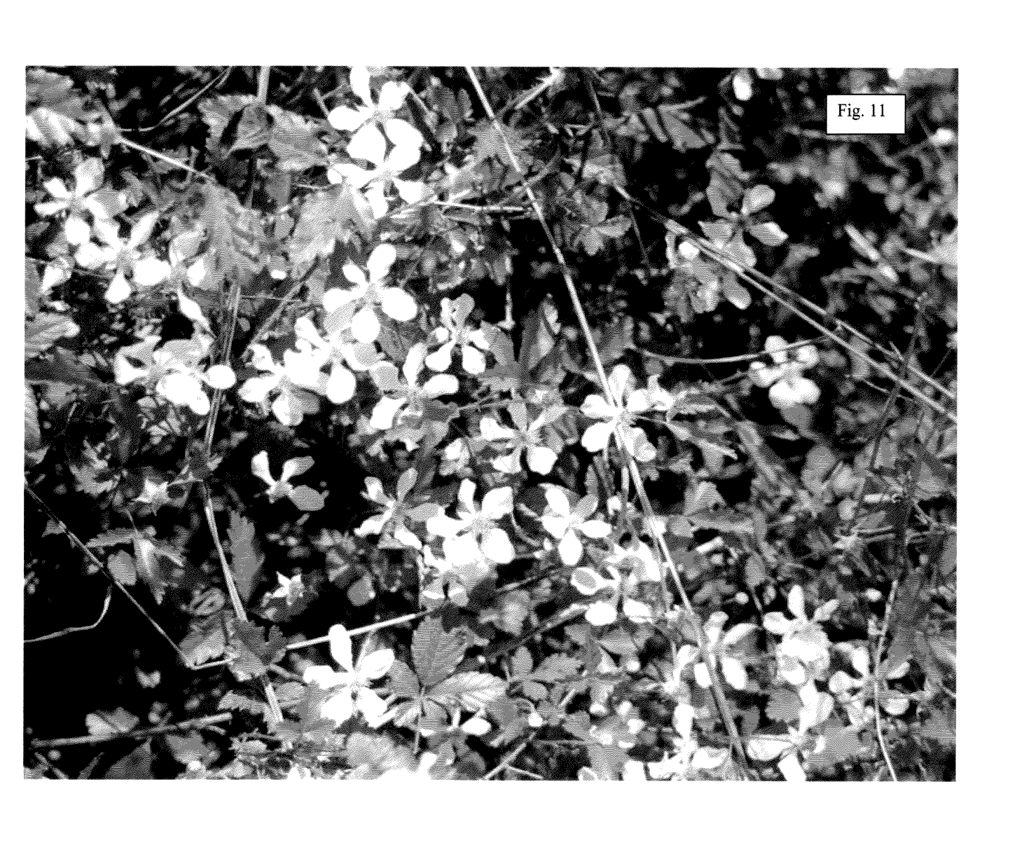

Fig.10 Wild blackberries—showing quantities of berries produced per each cane

We claim:

1. A new and distinctive variety of blackberry plant, substantially as described and illustrated herein, and is characterized by having a creamy white colored fruit which has the absence of anthocyanin coloration. The plant has many aspects like the wild blackberry.
The Latin name of the genus and species of the plant claimed: The variety is botanically identified as Rubus L. Subgenus Rubus.
